(19) United States
(12) Patent Application Publication
(10) Pub. No.: US 2024/0423686 A1
(43) Pub. Date: Dec. 26, 2024

(54) SLEEVE FOR SUPRAPATELLAR NAILING OF THE TIBIA

(71) Applicant: OrthoPediatrics Corp., Warsaw, IN (US)

(72) Inventors: Cristian Gurrola, Fort Wayne, IN (US); Chris M. Powers, Warsaw, IN (US); Luis Vega, Warsaw, IN (US)

(73) Assignee: OrthoPediatrics Corp., Warsaw, IN (US)

(21) Appl. No.: 18/750,745

(22) Filed: Jun. 21, 2024

Related U.S. Application Data
(60) Provisional application No. 63/509,611, filed on Jun. 22, 2023.

(51) Int. Cl. A61B 17/88

(52) U.S. Cl. CPC A61B 17/8872

(57) ABSTRACT

An apparatus for use with a tool during orthopedic surgery of a patient includes a flexible body defining a cannula and a flange. The body has an opened proximal end adapted and configured for receiving a tool. The body has an opened distal end adapted and configured for placement against the patient. The cannula has an axis. The flange is located at the proximal end and has at least a first ear extending outwardly away from the axis. The first ear includes a first aperture extending therethrough. The first aperture has an opened pathway extending generally orthogonally to the axis.

### CROSS-REFERENCE TO RELATED APPLICATION(S)

This application claims the benefit of priority of U.S. Provisional Patent Application No. 63/509,611, filed Jun. 22, 2023, the disclosure of which is incorporated herein by reference.

### FIELD OF THE INVENTION

Various embodiments of the present inventions relate to surgical instruments and, in particular, pertain to tools useful for the implantation of intramedullary nails.

### SUMMARY OF THE INVENTION

One or more embodiments provide an apparatus for use with a tool during orthopedic surgery of a patient includes a flexible body defining a cannula and a flange. The body has an opened proximal end adapted and configured for receiving a tool. The body has an opened distal end adapted and configured for placement against the patient. The cannula has an axis. The flange is located at the proximal end and has at least a first ear extending outwardly away from the axis. The first ear includes a first aperture extending therethrough. The first aperture has an opened pathway extending generally orthogonally to the axis.

### BRIEF DESCRIPTION OF THE DRAWINGS

Some of the figures shown herein may include dimensions. Further, the figures shown herein may have been created from scaled drawings, scaled models, or from photographs that are scalable. It is understood that such dimensions, or the relative scaling within a figure, are by way of example, and not to be construed as limiting unless so stated in a claim. Persons of ordinary skill will also recognize that computer-aided design (“CAD”) renderings may include lines that pertain to changes in the computer model, and not necessarily to component features.

FIG. 1 is a front, bottom, left side perspective CAD surface representation of an insertion sleeve according to one embodiment of the present invention.

FIG. 2 is a top, left side, frontal CAD line drawing of the device of FIG. 1.

FIG. 3A is a front elevational view of the apparatus of FIG. 1.

FIG. 3B is an orthogonal representation of the apparatus of FIG. 3A, showing a side elevational view of the left side.

FIG. 3C is a cross sectional representation of the apparatus of FIG. 3B as taken along line 3C-3C of FIG. 3B.

FIG. 4A is a view of the apparatus of FIG. 3A as taken along line 4A-4A.

FIG. 4B is a view of the apparatus of FIG. 3A as taken along line 4B-4B of FIG. 3A.

FIG. 5 is a side elevational view of a pin according to one embodiment of the present invention.

FIG. 6 is a side elevational view of the apparatus of FIG. 1 shown in conjunction with other orthopedic instruments used with the apparatus of FIG. 1.

FIG. 7A is a side perspective view of an assembly of assorted embodiments of the devices shown in FIG. 6.

FIG. 7B is a perspective CAD representation of the apparatus of FIG. 7A with the apparatus of FIG. 1 being coupled by pins to a femur according to one embodiment of the present invention.

FIG. 8A is a side perspective view of a pin within a radiolucent pin guide for use with the devices shown in FIG. 7A (or with the embodiments shown in FIG. 6).

FIG. 8B is a perspective CAD representation of the apparatus of FIG. 8A with the sleeve and tissue protector placed over the pin guide.

FIG. 9A shows a parallel pin guide inserted into a knee joint.

FIG. 9B shows the procedure illustrated in FIG. 9A with the parallel pin guide removed, and a pair of pins coupling the insertion sleeve to the femur.

### ELEMENT NUMBERING

The following is a list of element numbers used with all of the embodiments, and at least one noun used to describe that element. The “X” for all of these numbers is removed or replaced with a number (0 or greater) in the text and drawings of this application. Consistent with statements made elsewhere in this specification, these various 2-digit element numbers are used among multiple embodiments, and aspects of a particular element stated for one embodiment can be applied to the same element number in a different embodiment, except as shown and described differently, and as would be understood by a person of ordinary skill in the art. It is understood that none of the embodiments disclosed herein are limited to these nouns, and these element numbers can further include other words that would be understood by a person of ordinary skill reading and reviewing this disclosure in its entirety.

| | |
|---|---|
| 10 | tibia |
| 12 | patella |
| 14 | femur |
| 15 | |
| 20 | insertion sleeve |
| a | proximal end |
| b | distal end |
| 30 | body |
| 32 | wall |
| 34 | cannula |
| 35 | central axis |
| 36 | proximal end |
| 38 | distal end |
| 40 | flange |
| a | ear |
| b | thick section |
| c | thin section |
| 41 | proximal surface |
| 42 | pin apertures |
| a | length |
| b | width |
| c | height |
| d | angle at entrance and exit |
| e | pathway |
| 44 | periphery |
| 50 | pin |
| 52 | shaft |
| 54 | threaded end |
| 56 | driving end |
| 60 | tissue protector |
| 61 | sleeve |
| 62 | handle |
| 63 | central passage |
| a | proximal end |
| b | distal end |
| 66 | trocar |
| 67 | wire guide |
| a | proximal end |
| b | distal end |
| 68 | wire |
| 70 | radiolucent pin guide |
| 72 | parallel pin guide |

### DETAILED DESCRIPTION OF ONE OR MORE EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention, and further permits the reasonable and logical inference of still other embodiments as would be understood by persons of ordinary skill in the art.

It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “various embodiments” or “preferably,” refers to features and aspects that are present in at least one embodiment, but which are optional for some embodiments, it therefore being understood that use of the word “preferably” implies the term “optional.”

Although various specific quantities (spatial dimensions, temperatures, pressures, times, force, resistance, current, voltage, concentrations, wavelengths, frequencies, heat transfer coefficients, dimensionless parameters, etc.) may be stated herein, such specific quantities are presented as examples only, and further, unless otherwise explicitly noted, are approximate values, and should be considered as if the word “about” prefaced each quantity. Further, with discussion pertaining to a specific composition of matter, that description is by example only, and does not limit the applicability of other species of that composition, nor does it limit the applicability of other compositions unrelated to the cited composition.

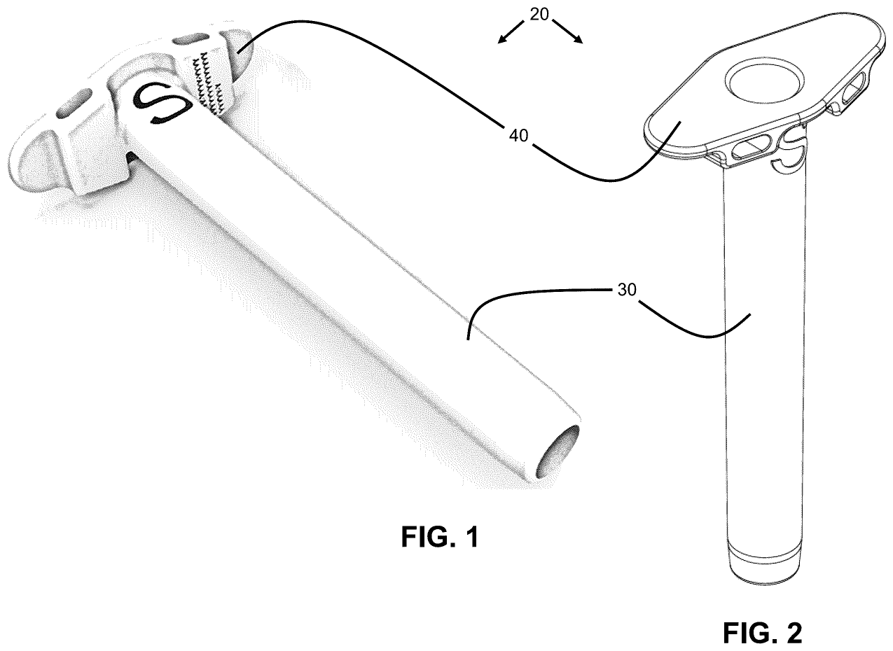

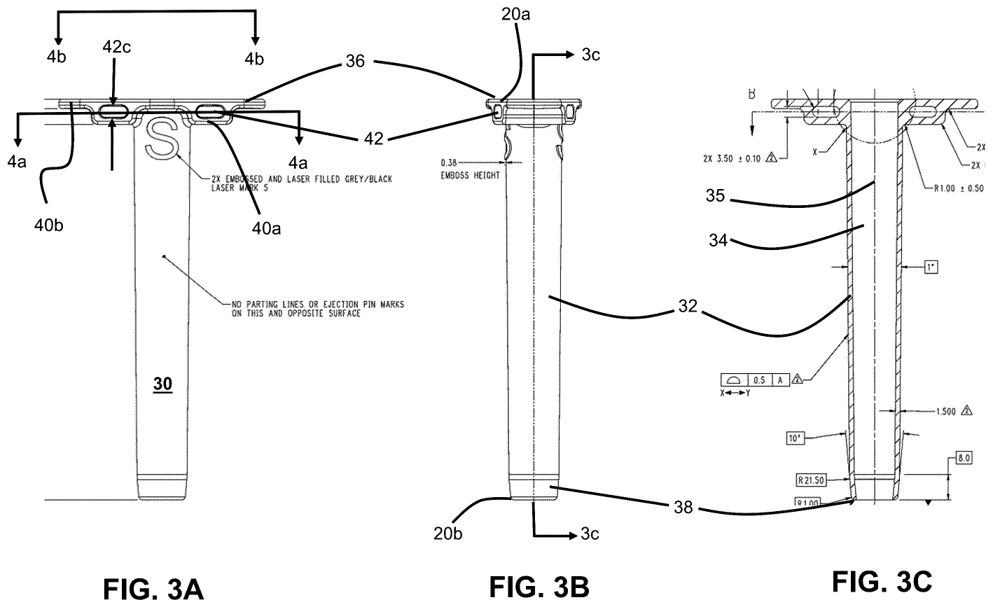

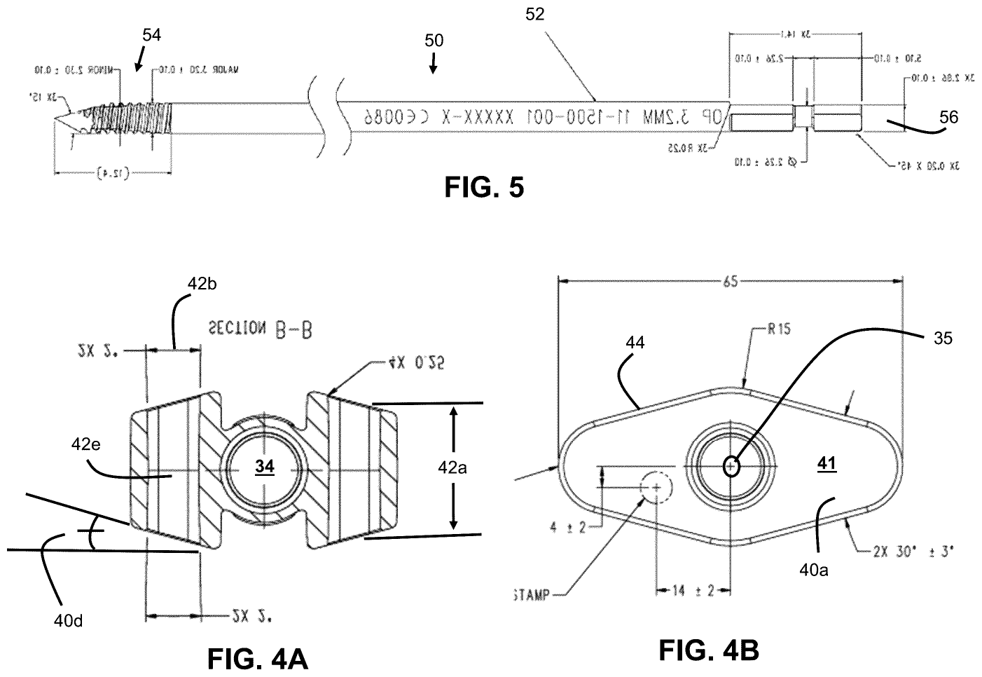

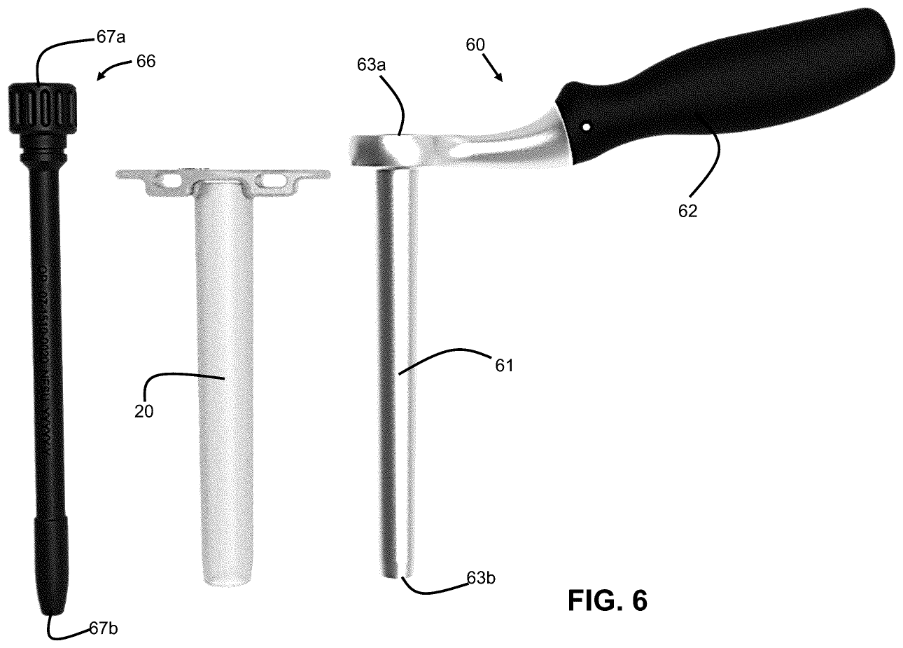

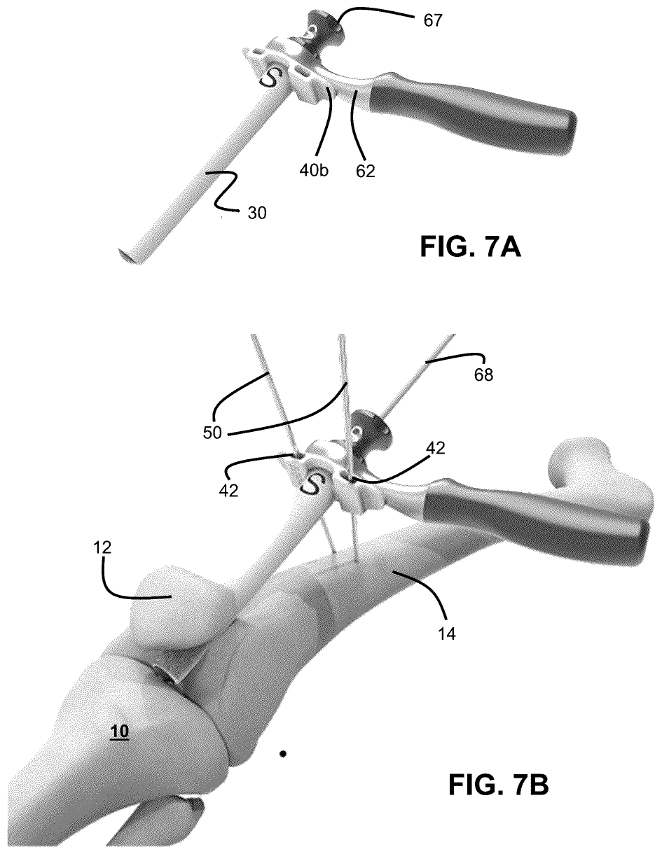

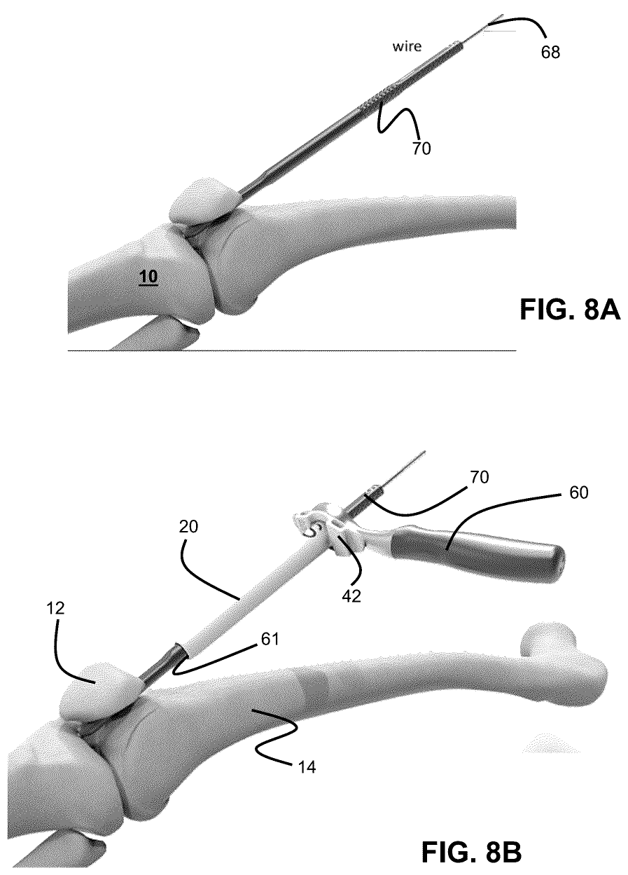

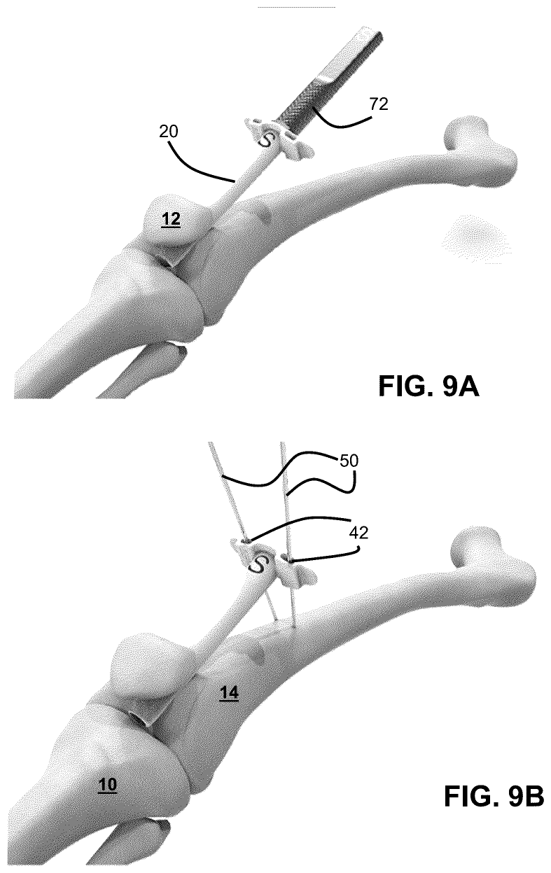

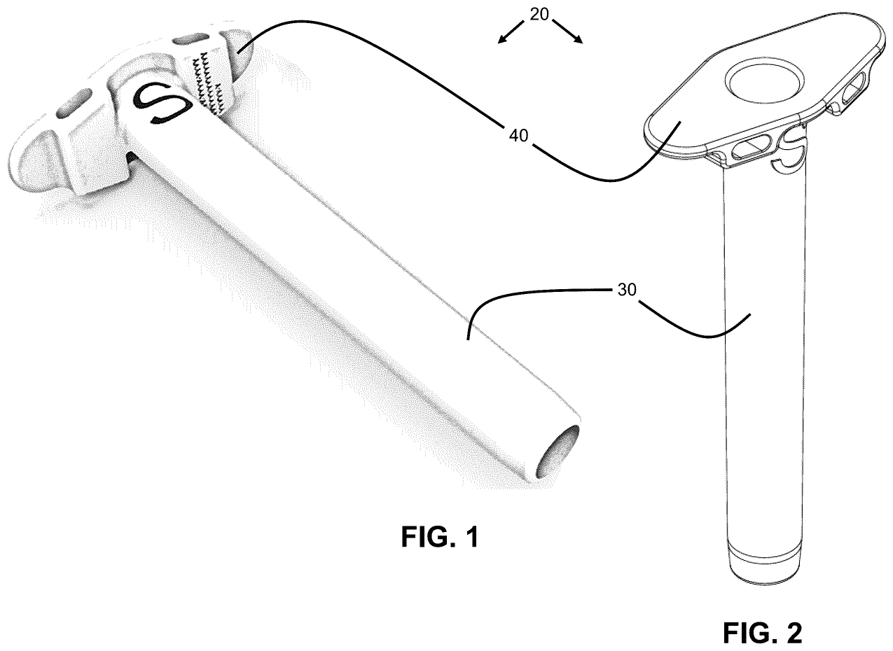

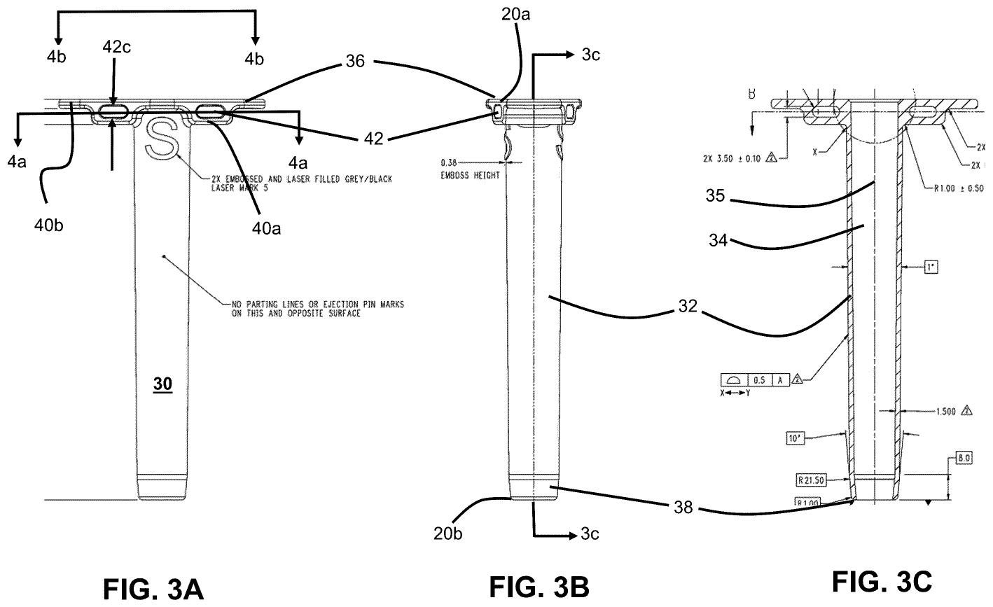

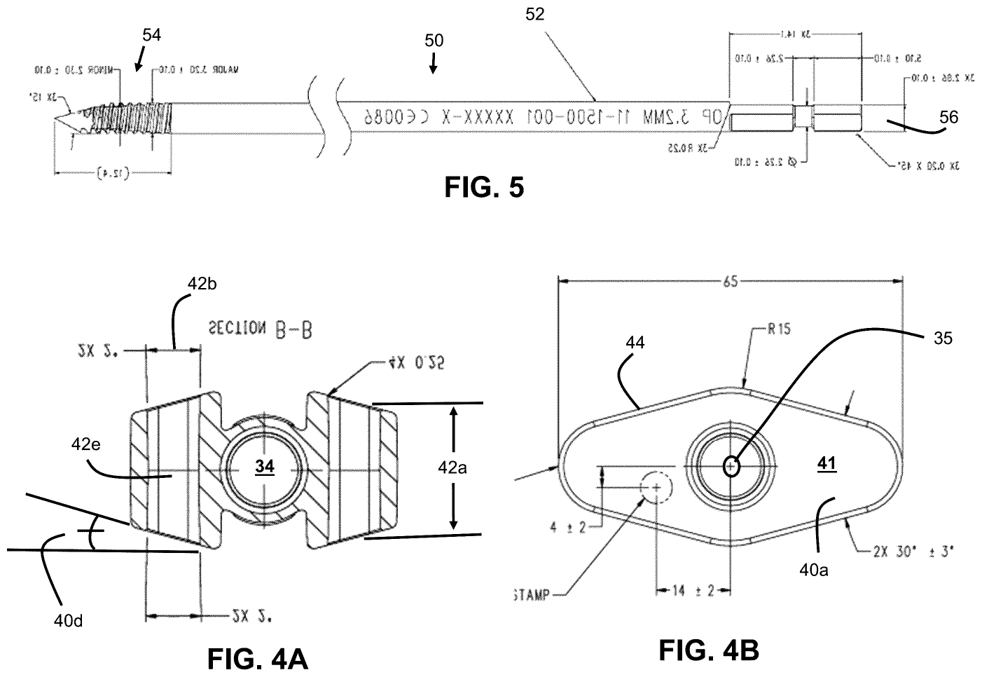

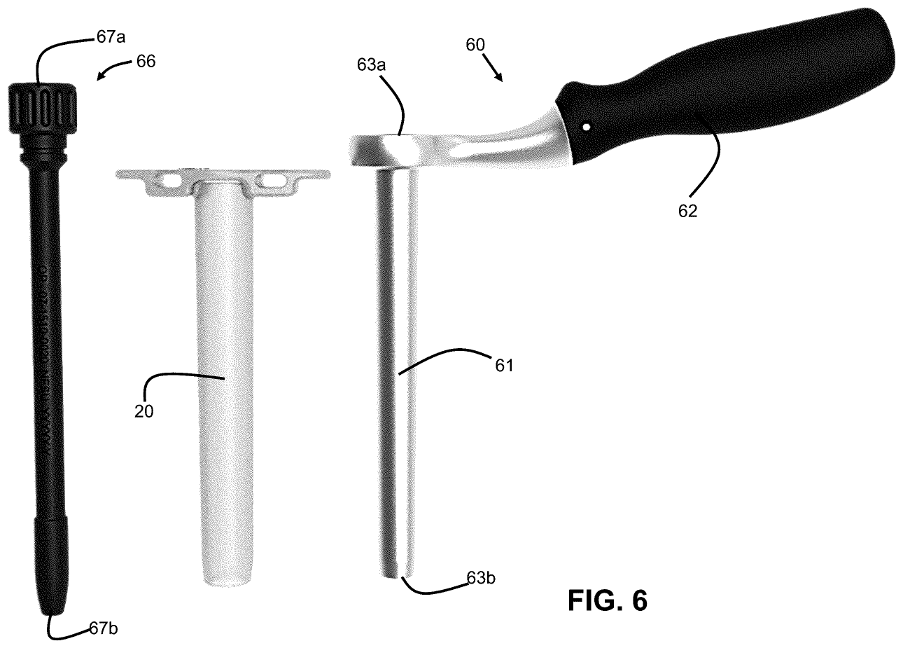

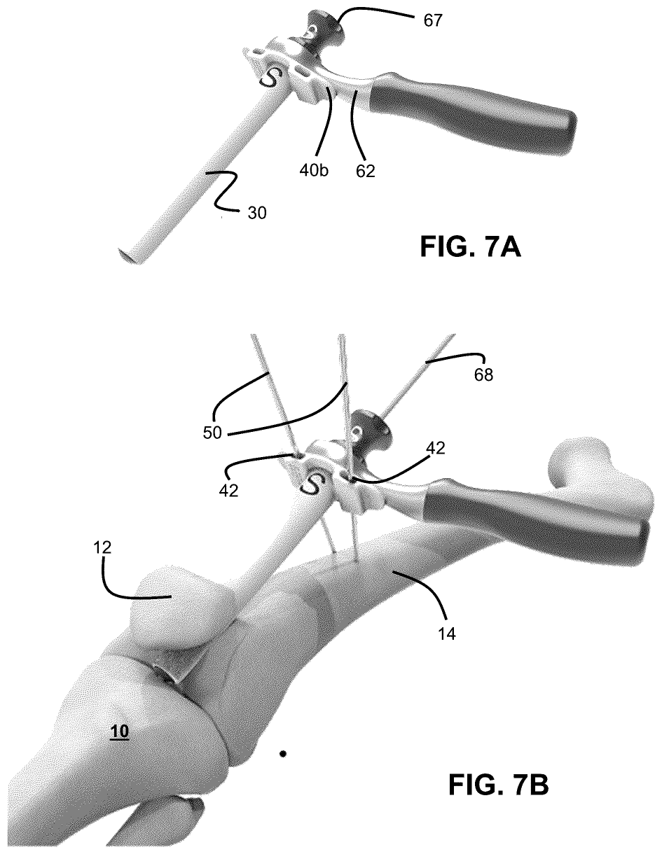

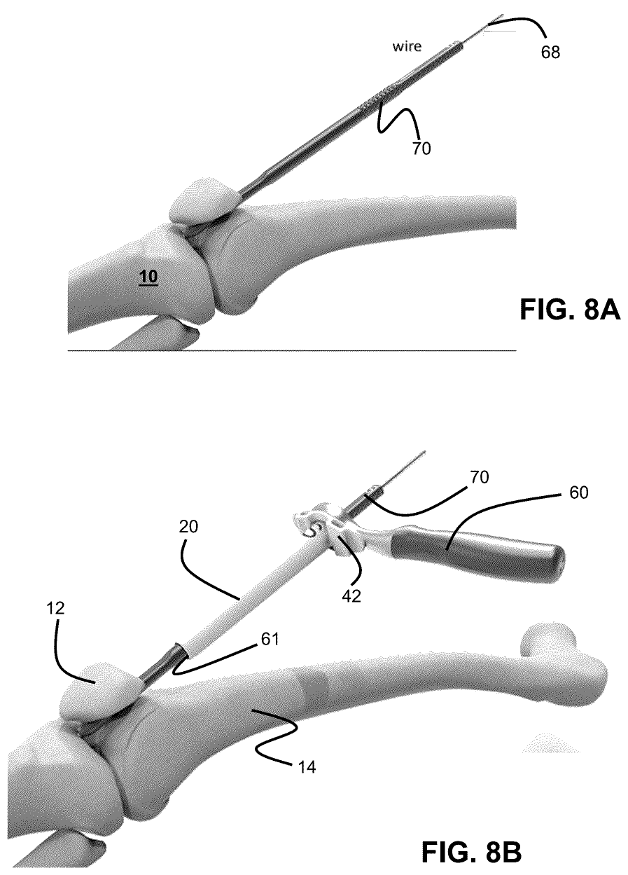

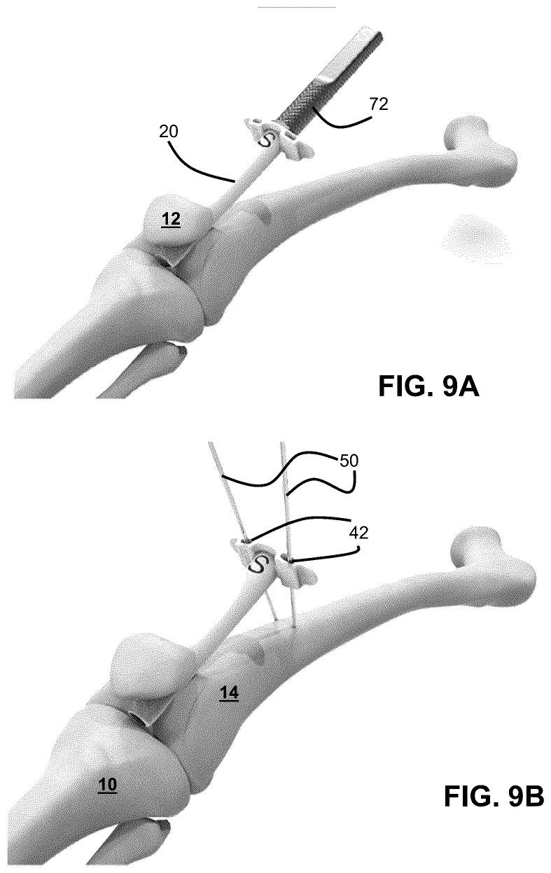

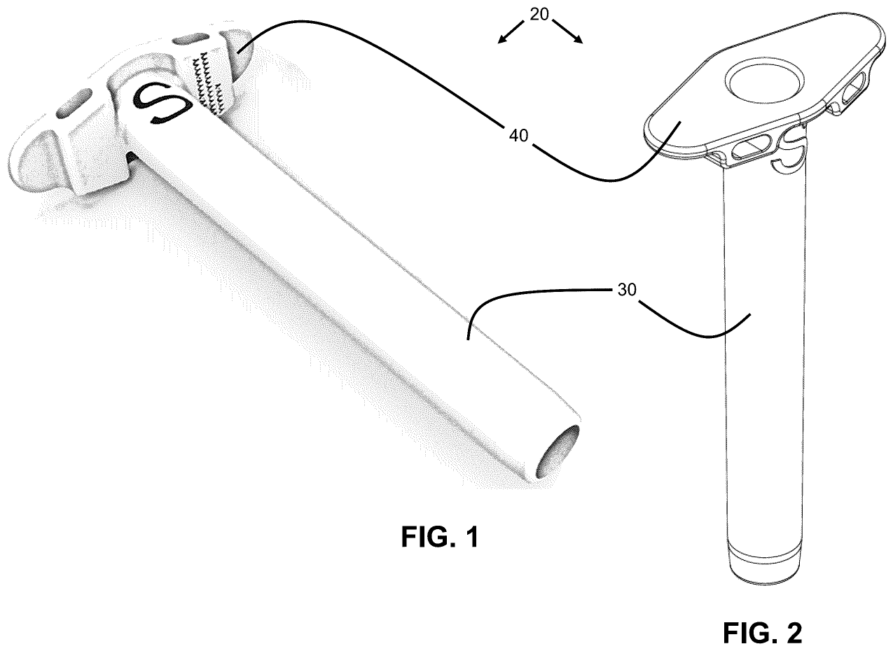

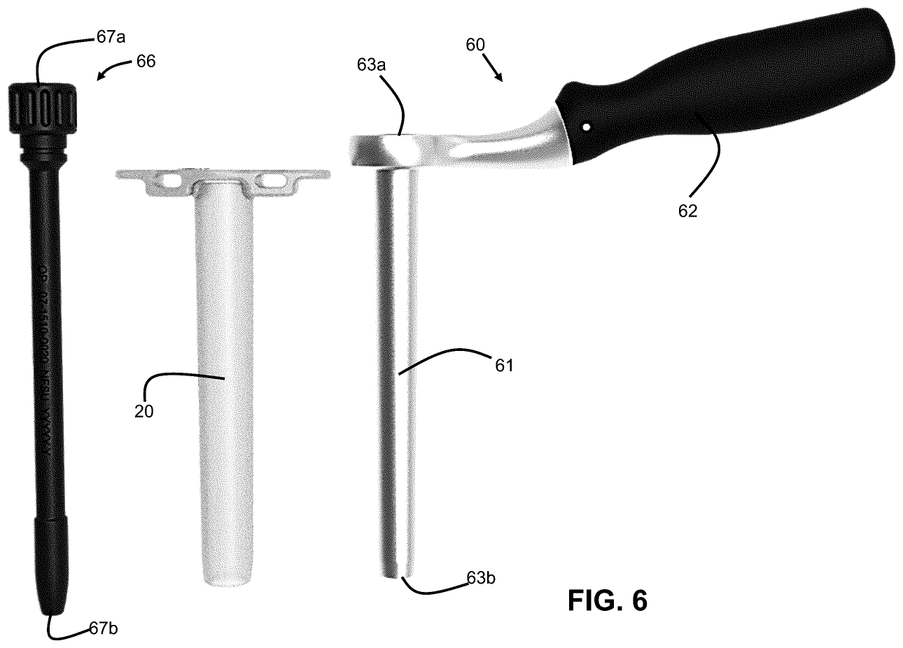

FIGS. 1-5 show various aspects of a soft insertion sleeve and pin which, in one embodiment of the present invention, are used during a procedure to insert an intramedullary nail into a tibia. FIGS. 6-9 depict at least three different options for using the sleeve in such a procedure. It is understood that the procedures depicted are by way of example only, and are not to be construed as limiting. Further, the sleeve and pin shown in FIGS. 1-5 can be used in orthopedic procedures, or other medical procedures, and are not limited to insertions of intramedullary nails.

FIGS. 1 and 2 show various perspective views of an insertion sleeve 20 according to one embodiment of the present invention. The sleeve 20 preferably includes a flange 40 that extends transversely from the distal end of a tubular body 30. In one embodiment, the insertion sleeve 20 is fabricated from a soft, flexible material such as Santoprene® TPV 8281-90MED (natural) or other soft, elastic biocompatible materials.

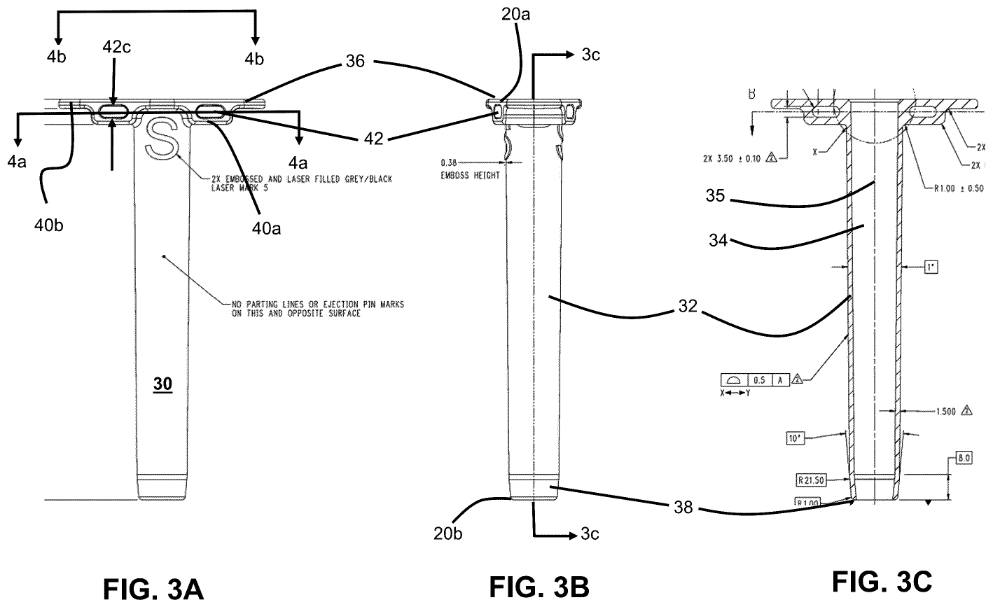

FIGS. 3A, 3B, and 3C show various views of the soft insertion sleeve 20. Sleeve 20 has a distal end 20a that includes a flange 40 with a body 30 that extends distally toward a distal end 20b. Flange 40 includes a pair of ears 40a that extend on opposite sides of body 30. Body 30 defines a cannula 34 that is adapted and configured to receive within it an instrument used in surgery, such as the orthopedic instruments shown in FIGS. 6-9. Preferably, cannula 34 is generally cylindrical, and extends along a central axis.

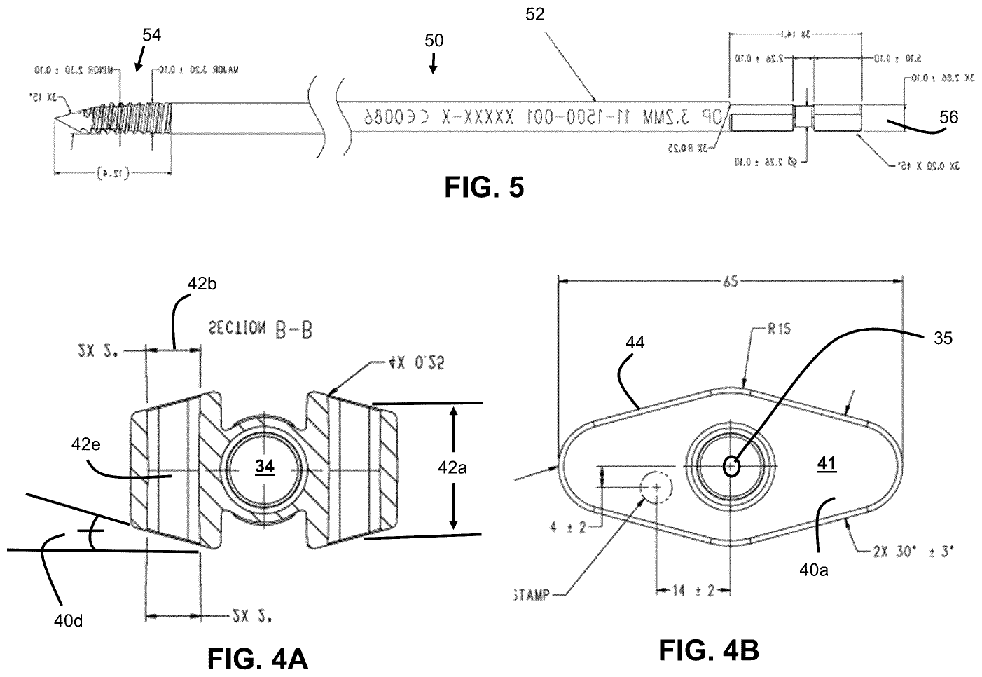

Referring to FIGS. 3A, 3B, 3C, 4A, and 4B, flange 40 includes a preferably planar top surface 41 having a perimeter 44 that is oval in shape, with each ear 40a having angled sides joined by a rounded portion. As best seen in FIG. 3A, flange 40 includes a pair of thicker sections 40b located on the distal side of each ear 40a and on opposite sides of body 30. A thin section 40b is located outboard of each of the thicker sections 40a. Each of the thicker sections 40a include pin apertures 42. In one embodiment, each aperture has the shape of an elongated oval, having a width 42b that is greater than the projected height 42c.

Referring to FIG. 4A, it can be seen that the apertures 42 are open on each end, the aperture ends providing entrance and exit to the internal pin pathway 40e, and each aperture extending along a length 42a that preferably traverses the entire width of the thick section 40a. In some embodiments, the entrance and exit include aperture faces 40d that are acutely angled relative to the centerline of the pathway 40e.

Referring to FIGS. 4A and 4B it can be seen that the flange is symmetric about both vertical and horizontal planes passing through the central axis 34. Apertures 42 are adapted and configured to provide clearance at the entrance, exit and pathway around an instrument such as the pin 50 shown in FIG. 5. The angulation of the exit and entrance relative to the pathway provide additional clearance at the entrance and exit for the pin, as well as permitting a wider range of pin angles in the pathway.

Pin 50 comprises a shaft 52 having a distal threaded end 54 and a proximal driving end 56. Threaded end 54 includes a sharp tip, and is adapted and configured for penetration into a bone. Driving end 56 is adapted and configured to mate with a tool (not shown) that can be used to rotate pin 50.

In some embodiments the aperture width 42b is preferably 2×-3× larger than the diameter of the pin, although it is understood that yet other embodiments of the present invention contemplate pin apertures that are of any size and shape, including apertures that more closely fit the pin diameter, and including circular or other shapes. In some embodiments, the height 42c of the pin aperture more closely fits the outer diameter of the pin, and in one embodiment the pin aperture height 42c is about 10 percent greater than the diameter of the pin. Although what has been shown and described is an aperture that provides clearance on all sides to a pin, it is further understood that yet other embodiments contemplate pin apertures that are more closely fitting to the pin.

FIG. 6 shows soft insertion sleeve 20 along with two instruments used in some medical procedures. Tissue protector 60 includes a handle 62 that is coupled to a sleeve 61, sleeve 61 being adapted and configured to fit within the cannula 34 of sleeve 20. Sleeve 61 has an opened cannula or central passage 63 that extends from a proximal end 63a to a distal end 63b. FIG. 6 further shows a wire guide 67 that can be placed within the central passage 63 of tissue protector 60. Preferably, wire guide 67 further includes an opened central passage that in some embodiments receives another tool such as a wire or pin. Although what has been shown and described are certain instruments or tools being used with one embodiment of the sleeve, it is understood such examples are non-limiting, and yet other instruments and tools can be used with the sleeve.

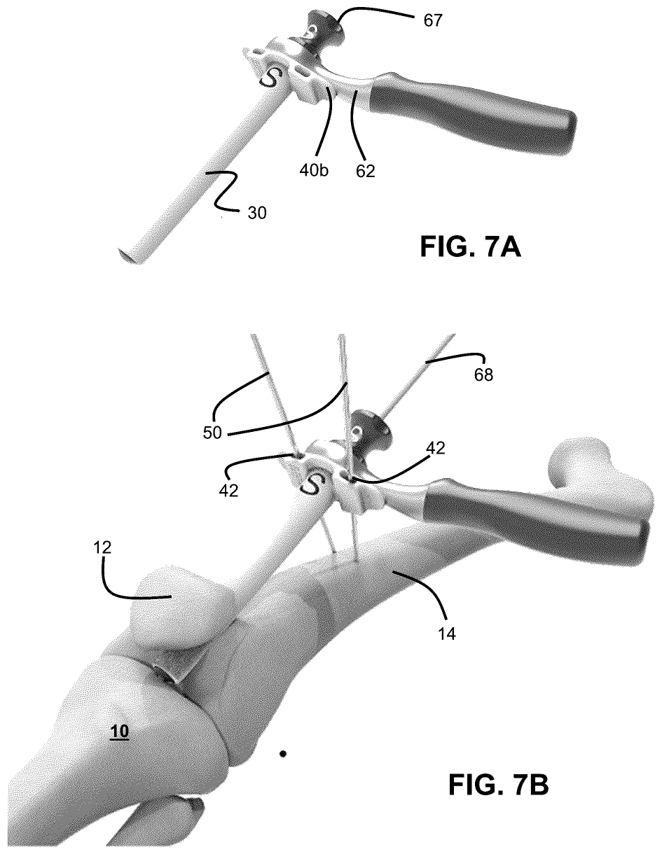

FIGS. 7A, 7B, 8A, 8B, 9A, and 9B show, respectively, three different examples of medical procedures that can utilize the insertion sleeve 20. FIGS. 7A and 7B show a first procedural option for use of sleeve 20. FIG. 7A shows an assembly of assorted embodiments of the instruments shown in FIG. 6, with the wire guide 67 being inserted within the central passage 63 of tissue protector 60, and with the sleeve 61 being inserted into the cannula 44 of sleeve 20.

FIG. 7B shows the assembly of FIG. 7A during one point in the orthopedic procedure. The assembly has been placed in contact with the top surface of tibia 10 in a suprapatellar approach in which the external surface of body 30 protects both the patella 12, femur 14, as well as other parts of the knee joint from abrasion by the instruments. A pair of pins 50 have been placed through corresponding pin apertures 42, and threaded into the femur, from which position the pins limit any movement of the sleeve (as well as the accompanying instruments) relative to the femur. FIG. 7B further shows a wire 68 that is inserted through the central passage of wire guide 67. After the pins are threaded into the femur, the tissue protector is free to rotate within the pinned insertion sleeve 20.

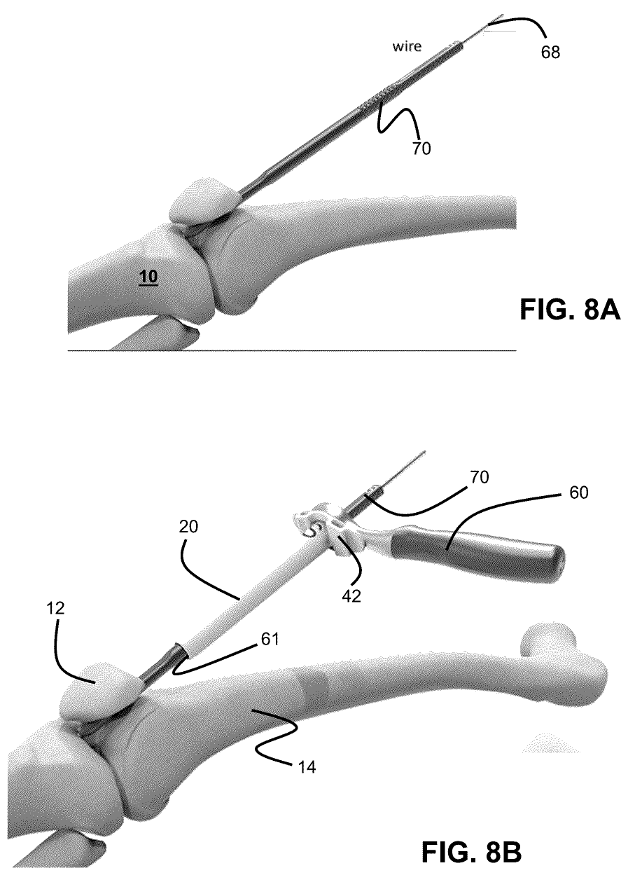

FIGS. 8A and 8B present another option for use of sleeve 20 in an orthopedic procedure, such as a procedure for insertion of an intramedullary nail into the tibia. FIG. 8A shows a radiolucent pin guide 70 placed between the patella and femur, with the distal end pin guide being in contact with the tibia. A wire 67 extends through a cannula within the pin guide, and into tibia 10. FIG. 8B shows a further step within that procedure, in which the assembly of the insertion sleeve 20 and tissue protector 60 have been married together, and placed around the pin guide 70. FIG. 8B shows the assembly of sleeve 20 and protector 60 prior to placement of sleeve 20 between the patella and femur.

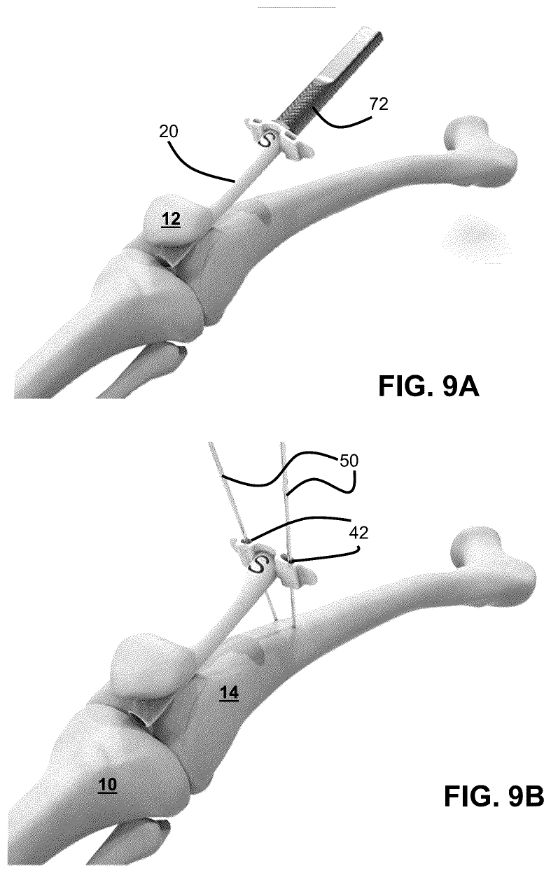

FIGS. 9A and 9B show yet another option for use of the insertion sleeve 20 within an orthopedic procedure. FIG. 9A shows sleeve 20 supporting within it a parallel pin guide 72. This assembly has been placed between the patella and tibia, with the distal end of the assembly being in contact with the tibia. FIG. 9B shows a further step within this procedure, in which a pair of pins 50 have been placed within corresponding apertures 42, with the threaded end of the pins being threaded into the femur.

While the invention(s) has (have) been illustrated and described in detail in the drawings and foregoing description, the same is to be considered as illustrative and not restrictive in character, it being understood that only certain embodiments have been shown and described and that all changes and modifications that come within the spirit of the invention(s) are desired to be protected.

What is claimed is:

1. An apparatus for use with a tool during orthopedic surgery of a patient, the apparatus comprising:
a flexible body defining a cannula and a flange, the body having an opened proximal end adapted and configured for receiving a tool, the body having an opened distal end adapted and configured for placement against the patient, the cannula having an axis, the flange being located at the proximal end and having at least a first ear extending outwardly away from the axis, the first ear including a first aperture extending therethrough, the first aperture having an opened pathway extending generally orthogonally to the axis.

2. The apparatus of claim 1, wherein the flange has a second ear including a second aperture extending generally parallel to the first aperture.

3. The apparatus of claim 2, wherein the first ear and the second ear are located on opposite sides of the body.

4. The apparatus of claim 3, wherein the first aperture has an oval cross-sectional shape.

5. The apparatus of claim 4, wherein the cannula is cylindrical.

6. The apparatus of claim 5, further comprising:
a threaded pin having a diameter and adapted and configured for threaded connection to a bone,
wherein the first aperture has a width and the width is more than about twice the diameter of the pin.

7. The apparatus of claim 1, further comprising:
a threaded pin having a diameter and adapted and configured for threaded connection to a bone,
wherein the first aperture has a width and the width is more than about twice the diameter of the pin.

8. The apparatus of claim 3, further comprising:
a threaded pin having a diameter and adapted and configured for threaded connection to a bone,
wherein the first aperture has a width and the width is more than about twice the diameter of the pin.
